(19) United States
(12) Patent Application Publication
(10) Pub. No.: US 2019/0217006 A1
(43) Pub. Date: Jul. 18, 2019

(54) FIBER OPTICALLY ILLUMINATED MEDICAL INFUSION TUBES

(71) Applicants: Ananya ANAND, Los Altos Hills, CA (US); Naya ABHIRAM, Seattle, WA (US); Namrata ANAND, Los Altos Hills, CA (US)

(72) Inventors: Ananya ANAND, Los Altos Hills, CA (US); Naya ABHIRAM, Seattle, WA (US); Namrata ANAND, Los Altos Hills, CA (US)

(21) Appl. No.: 16/239,802

(22) Filed: Jan. 4, 2019

Related U.S. Application Data
(60) Provisional application No. 62/617,216, filed on Jan. 13, 2018.

(51) Int. Cl. A61M 5/142; A61B 1/07

(52) U.S. Cl. CPC A61M 5/142; A61M 5/365; A61B 1/07

(57) ABSTRACT

Fiber optically illuminated medical infusion tubes. Such an illuminated tube includes one or more infusion tubes, one or more optical fibers which is illuminated for the infusion tube and one or more light sources which lights up the optical fiber. The fiber is illuminated along the length of the infusion tube. The light sources have multiple light colors to identify each of the optical fiber. A light controller controls the lightings of the optical fiber, and the light controller can be programed for various lighting modes as desired.

### CROSS-REFERENCE TO RELATED APPLICATIONS

This application claims priority from U.S. Provisional Patent Application No. 62/617,216, filed on Jan. 13, 2018, entitled “Fiber Optically Illuminated Medical Infusion Tubes,” the entire contents of which are hereby incorporated by reference.

### BACKGROUND

Modern day fluid infusion systems have made the ability of medical practitioners to deliver fluids, medicines, drugs in a systematic, programmatic and controlled manner much more easily and efficiently. These systems allow staff to maintain sterile environments, program the infusion controllers and pumps for rate and volume of delivery, start and end times, and be alerted in case of completion of fluid delivery or blockages.

The infusion tubes are manufactured and shipped in sterile packages for single use. These tubes also have a lot of flexibility to be linked together, chained, with ports and connectors to enable many pumps; and hence multiple drugs/medicines can be simultaneously delivered through a single entry line, either peripheral or central, directly into the vascular system of a patient.

Uniquely identifying tubes when multiple infusion tubes are connected to a single port/line of a patient is generally done by putting sticker labels with text written on them that identify the medicine, dosage information, drug interaction risks, tube start use day and time, tube expiry date and time and any other relevant information. This process restricts the ability of staff to trace a specific tube and/or medicine with a twisted nest of tubes under time constraints, in poor lighting conditions, and in restricted physical conditions, making this aspect of patient care tedious, time-consuming and error prone.

There may also be colored infusion tubes, in a variety of colors, each associated with a different class of drug such as antibiotics, pain medicines, narcotics, etc. The challenge with such an approach is managing inventory, supplies for a variety of tube colors, the inability to determine usage and expiry times of a tube, uniquely identify a specific infused medicine, or identify an alerted infusion that is either complete or kinked or blocked. Also, if certain colors of tubes have to be used for certain types of drugs, there is a possibility of making errors by picking a wrong tube color for an infusion.

### SUMMARY

According to at least one exemplary embodiment, fiber optically illuminated medical infusion tubes may be described. Such an illuminated tube may include: one or more infusion tubes; one or more optical fibers which is illuminated for the infusion tube; and one or more light sources which lights up the optical fiber. In an exemplary embodiment, the fiber may be illuminated along the length of the infusion tube. Also, the light sources may have multiple light colors to identify each of the optical fiber. Further, a light controller may control the lightings of the optical fiber, and the light controller may be programed for various lighting modes as desired.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1A is a cross sectional view of an exemplary embodiment where an optical fiber is incorporated into the length of an infusion tube.

FIG. 1B is another cross sectional view of an exemplary embodiment where an optical fiber is incorporated into the length of an infusion tube.

FIG. 1C is another cross sectional view of an exemplary embodiment where an optical fiber is incorporated into the length of an infusion tube.

FIG. 1D is another cross sectional view of an exemplary embodiment where an optical fiber is incorporated into the length of an infusion tube.

FIG. 1E is another cross sectional view of an exemplary embodiment where an optical fiber is incorporated into the length of an infusion tube.

FIG. 1F is another cross sectional view of an exemplary embodiment where an optical fiber is incorporated into the length of an infusion tube.

FIG. 2A is an exemplary embodiment of a light source adapter.

FIG. 2B is another exemplary embodiment of a light source adapter.

FIG. 3 is an exemplary embodiment of a fiber-optic embedded infusion tube with the switchable light adapter.

FIG. 4 is an exemplary embodiment of a switchable light source incorporated into an infusion pump/controller.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

According to an exemplary embodiment, and referring generally to the Figures, various exemplary implementations of fiber optically illuminated medical infusion tubes may be disclosed. In an exemplary embodiment, optical fibers may directly be embedded into infusion tubes and illuminated in a potential of one of multiple colors using light sources such as LEDs to uniquely identify each infusion tube, rapidly and in error free manner when there are a multitude of infusion tubes connected to a patient using multiple infusion controllers, pumps and gravitational flow mechanisms.

According to an exemplary embodiment, a light in one of many colors may be used to light up an infusion line either manually, remotely, programmatically or automatically as desired. In an exemplary embodiment, an optical fiber may be embedded on the exterior of infusion tubes as a mechanism to transmit light along the whole length of the tube. Also, in an exemplary embodiment, a color-selectable light source at a point close to the source of the fluid may be either integrated into the controller/pump or attached at the pump. The light source may be capable of lighting up the entire length of the infusion tube from the pump exit point till the exit point of the tube, which may be at a connector, joint, or into the infusion port of the patient. The light may be static in one color, with a programmed flash duration in one color, with a programmed flash sequence in one color or in a variety of changing colors identifying the infusion tube for a drug type, drug infusion completion, pump error condition, tube kink, blockage issue or etc.

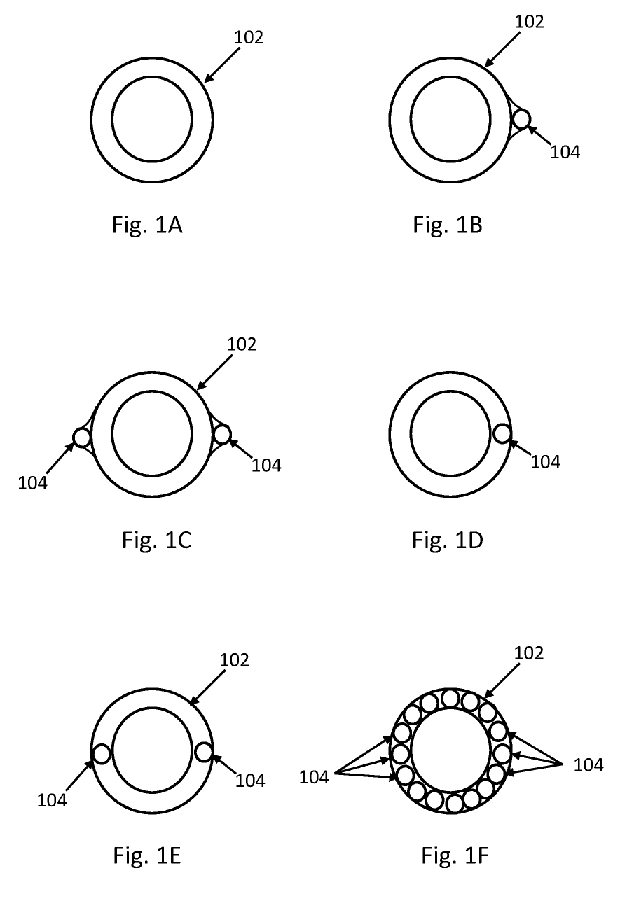

Turning now to exemplary FIGS. 1A-1F, FIGS. 1A-1F display cross sectional views of exemplary embodiments where an optical fiber is incorporated into the length of an infusion tube. According to an exemplary embodiment, plastic infusion tubes 102 are integrated with optical fibers 104 in various ways so that they may transmit light of any color, solid or patterned pulsing or changing colors along the whole length of the infusion tube 102.

Referring still to FIGS. 1A-1F, FIG. 1A shows the cross-sectional view of a normal flexible plastic infusion tube 102. FIG. 1B shows an exemplary embodiment of plastic infusion tube 102 with the optical fiber 104 adhered and embedded on the outer surface along the length of the infusion tube 102. FIG. 1C shows an exemplary embodiment of plastic infusion tube 102 with the optical fibers 104 adhered and embedded on the outer surface along the length of the infusion tube 102 at diametrically opposite sides of the tube 102. FIG. 1D shows an exemplary embodiment of plastic infusion tube 102 with the optical fiber 104 embedded within the physical structure of tube 102. FIG. 1E shows an exemplary embodiment of plastic infusion tube 102 with the optical fibers 104 embedded within the physical structure of tube 102 on diametrically opposites of the tube 102. FIG. 1F shows an exemplary embodiment of plastic infusion tube 102 with the optical fibers 104 embedded within the physical structure of tube 102 in an annular ring fashion around circumference of the whole infusion tube 102.

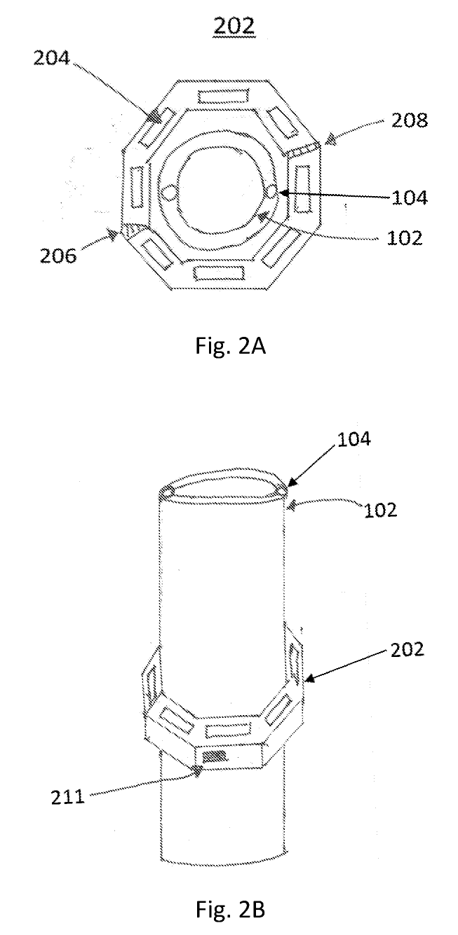

Turning now to exemplary FIGS. 2A and 2B, FIGS. 2A and B show an exemplary embodiment of a light source adapter. According to an exemplary embodiment, the light source adapter 202 may light up the optical fiber 104 integrated infusion tube 102. Also, in an exemplary embodiment, the light source adapter 202 may include an incorporated battery and may light up the optical fiber 104 in cycling through multiple colors of lights or set of on many pulsing sequences including a steady light. According to an exemplary embodiment, the adapter 202 may be capable of being attached to one of many embodiments of the optical fiber 104 integrated infusion tube 102 at the point of tube exiting an infusion pump to either side of the pump, fluid side and/or patient side. Referring to FIG. 2A, the light source adapter 202 may include many light color sources 204 such as LEDs. Also, in an exemplary embodiment, the adapter 202 may have a hinge 206 and a latch-plus-lock 208 that may allow the adapter 202 to be opened or encircle the optical fiber 104 integrated fusion tube 102 and then locked. Also, FIG. 2A shows the cross-sectional view of the optical fiber 104 integrated infusion tube 102 within the adapter 202.

Referring to FIG. 2B, FIG. 2B shows the side view of the light source adapter 202, with a control switch 211 that may allow various functions of the adapter 202, for example, being turned ON, cycled through colors, steady and pulsed modes. In addition, FIG. 2B shows the optical fiber 104 integrated infusion tube 102 running through the adapter 202.

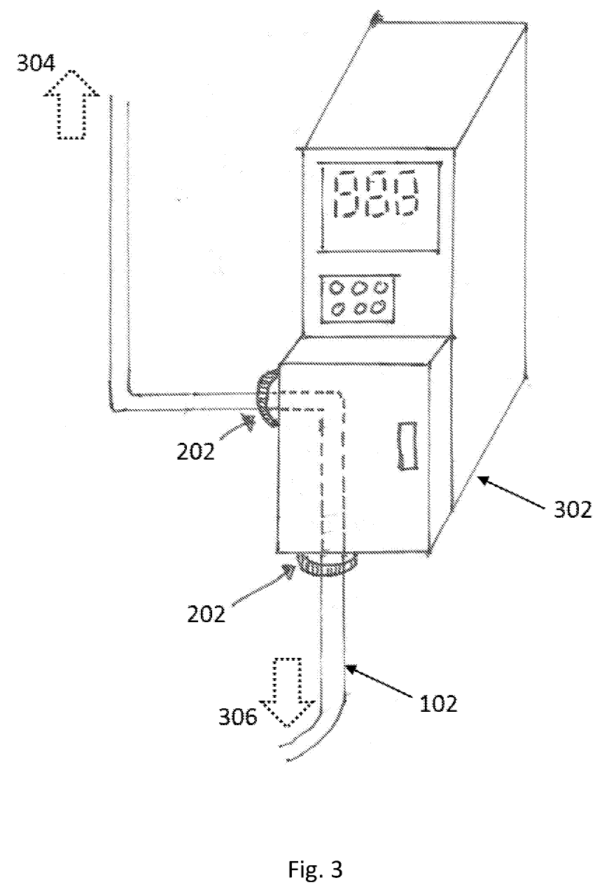

Turning now to exemplary FIG. 3, FIG. 3 shows an exemplary embodiment of a fiber-optic embedded infusion tube with the switchable light adapter attached to an infusion controller and pump. According to an exemplary embodiment, through an infusion controller-pump 302, the optical fiber 104 integrated infusion tube 102 may be guided. In an exemplary embodiment, more than one of the light source adapter 202 may be attached around the infusion tube 102 on both sides of the infusion controller/pump 302: fluid side 304; and patient side 306 so that nursing and patient care staff may light up the infusion tube 102 in the color they choose.

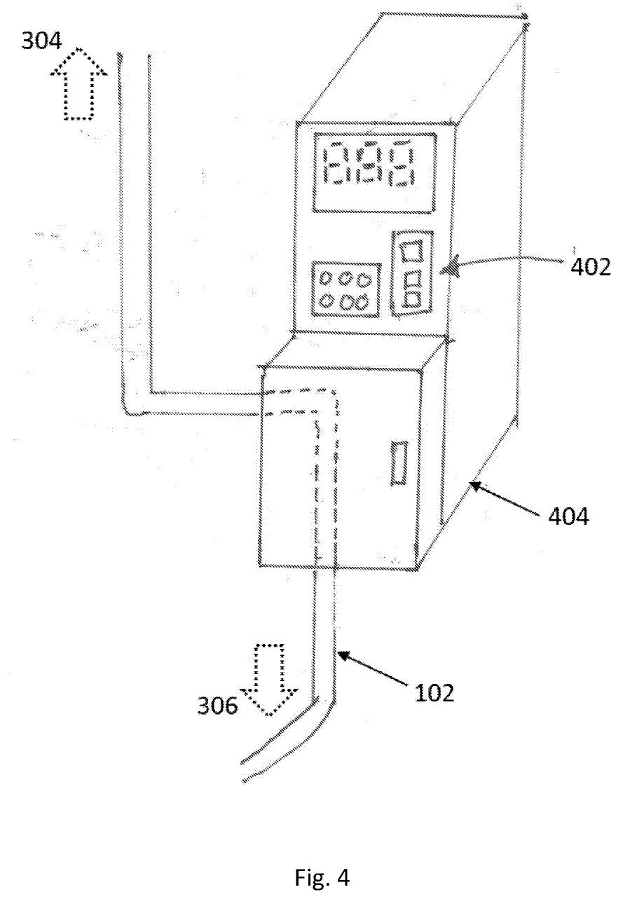

Turning now to exemplary FIG. 4, FIG. 4 shows an exemplary embodiment of a switchable light source incorporated into an infusion pump-controller. According to an exemplary embodiment, the functions of the light source adapter 202 may be integrated into an enhanced infusion pump/controller 404 through which the optical fiber 104 integrated infusion tube 102 is routed with a fluid side 304 and patient side 306, and a user may control the lighting via a lighting control panel 402 on the pump-controller 404 or by a remote controller. In an exemplary embodiment, the enhanced infusion pump-controller 404 may provide various functions for nursing and care staffs, for example: lighting up the infusion tube 102 in a specific color on the fluid side 304 or patient side 306, either simultaneously, or singularly, in the same or different colors, using the lighting control panel 402 or remotely; allowing the controller 404 to be controlled remotely through a wired or wireless network; programing the controller 404 to light up patient side 306 of the infusion tube 102 upon completion of infusion; programing the controller 404 to light up with an error flashing pattern on either fluid side 304 or patient side 306 when a blockage, kink or air-bubble is detected by the pump 404; programing the controller 404 to light up infusion tube 102 in an unique color based on the infusion with different colors for antibiotics, pain-medicines or other categories of drugs and fluids; programing the controller 404 to light up the infusion tube 102 at the starting point of a timed infusion event; programing an array of controllers/pumps 404 to light up by type of medicine or by infusion tube usage expiry time or other conditions; and lighting up each infusion tube uniquely to allow nursing or care staff to identify a medicine change or disconnect an infusion, or debug an infusion error.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. Illuminated medical infusion tube comprising:
at least one infusion tube;
at least one optical fiber configured to be illuminated for the at least one infusion tube; and
at least one light source configured to light up the at least one optical fiber.

2. The illuminated tube of claim 1, wherein the at least one infusion tube is a flexible tube.

3. The illuminated tube of claim 1, wherein the at least one optical fiber is illuminated along a length of the at least one infusion tube.

4. The illuminated tube of claim 1, wherein the at least one optical fiber is adhered on a surface of the at least one infusion tube.

5. The illuminated tube of claim 1, wherein the at least one optical fiber is embedded within the at least one infusion tube.

6. The illuminated tube of claim 1, wherein the at least one light source includes a plurality of light colors to light up and identify the at least one optical fiber.

7. The illuminated tube of claim 1 further comprises a light controller, and the light controller is configured to control lightings of the at least one optical fiber and be programed for a plurality of lighting modes.

8. The illuminated tube of claim 7, wherein the plurality of lighting modes includes at least one of a steady lighting mode, a cycling through colors mode, a pulse lighting mode, an error flashing mode, a tube usage condition alarm mode and a tube usage expiry time alarm mode.

9. The illuminated tube of claim 7 further comprises at least one light source adapter, wherein the at least one light source adapter includes the at least one light source and the light controller and is attached to the at least one infusion tube.

10. The illuminated tube of claim 9, wherein the at least one light source adapter includes a hinge and a lock and is configured to be opened, encircled the at least one infusion tube and locked.

11. The illuminated tube of claim 9, wherein the at least one light source adapter includes a switch by which a user controls lighting of the at least one optical fiber.

12. The illuminated tube of claim 9, wherein the at least one light source adapter is configured to control lighting of the at least one optical fiber via the light controller.

13. The illuminated tube of claim 9, wherein the at least one light source adapter is configured to be controlled remotely.

14. The illuminated tube of claim 9 further comprises at least one infusion pump-controller and, wherein the at least one light source adapter is attached on the at least one infusion tube at least one of sides of the at least one infusion pump-controller: a fluid side; and a patient side.

15. The illuminated tube of claim 7 further comprises at least one infusion pump-controller and, wherein the at least one infusion pump-controller includes the at least one light source and the light controller.

16. The illuminated tube of claim 15, wherein through the at least one infusion pump-controller, the at least one infusion tube is routed with a fluid side and a patient side of the at least one infusion pump-controller.

17. The illuminated tube of claim 15, wherein the at least one infusion pump-controller is configured to be controlled with a control panel on the at least one infusion pump-controller.

18. The illuminated tube of claim 15, wherein the at least one infusion pump-controller is configured to control lighting of the at least one optical fiber via the lighting controller.

19. The illuminated tube of claim 15, wherein the at least one infusion pump-controller is configured to be controlled remotely.

20. A method of illuminating medical infusion tube comprising:
lighting up, by at least one light source, at least one optical fiber of at least one infusion tube,
wherein the at least one optical fiber is configured to be illuminated identifying the at least one infusion tube.
